(19) United States
(12) Patent Application Publication
(10) Pub. No.: US 2004/0260185 A1
(43) Pub. Date: Dec. 23, 2004

(54) Method of cardiac risk assessment

(75) Inventors: Stephen T. Anderson, North Oaks, MN (US); Dean J. MacCarter, Englewood, CO (US)

Correspondence Address: NIKOLAI & MERSEREAU, P.A., 900 SECOND AVENUE SOUTH, SUITE 820, MINNEAPOLIS, MN, 55402, US

(73) Assignee: Cortex Biophysik GmbH, Leipzig (DE)

(21) Appl. No.: 10/891,752

(22) Filed: Jul. 12, 2004

Related U.S. Application Data
(63) Continuation of application No. PCT/US03/13281, filed on Apr. 29, 2003.
Application No. PCT/US03/13281 is a continuation-in-part of application No. 10/138,442, filed on May 3, 2002.

(51) Int. Cl. A61B 5/02; A61B 5/00; A61B 5/08

(52) U.S. Cl. 600/481; 600/300; 128/920; 600/529

(57) ABSTRACT

A method of data management for assessing a patient's autonomic balance, risk of death, and the patient's response to therapy in terms of these assessments is described. This method describes a process by which a set of “raw variables” (RV) are translated into one or more of a new variable, defined as an Mortality Prediction Index, (MPI) that quantifies the patient's cardiovascular reflex control and risk of death. The translated variables are representative of both central and peripheral chemo receptivity, baroreflexes, and peripheral ergo receptors, which, in turn, provide the measurement of sympathovagal, or autonomic, balance. The process of selection and measurement of the MPI, and thus the sympathetic and parasympathetic components of autonomic balance at rest and during dynamic, isotonic exercise and recovery is described. The invention will further define risk of death using a Kaplan-Meier Plot for certain translated variables. The method will enable physicians to collect, view, track and manage complicated data from multiple sources using simple, well-understood visualization techniques to better understand the consequences of their therapeutic actions.

### BACKGROUND OF THE INVENTION

I. Field of the Invention

The present invention relates generally to the field of data management and data processing. More particularly, the invention involves the management and processing of patient data for assessing a patient's autonomic balance, risk of death and a patient's response to therapy. The disclosed method enables physicians to collect, view, track and manage complicated data from multiple sources using simple, well-understood visualization techniques to better understand the consequences of therapeutic actions. Data provided includes, but is not limited to, dynamic-cardiopulmonary variables (DCP) measured using a cardiopulmonary exercise (CPX) testing system and static, biochemical/neurohumoral variables (SBNV) collected from available laboratory blood chemistry instrumentation.

II. Related Art

It is well recognized that monitoring a patient's physiologic condition using computerized systems is valuable. For this reason, a wide variety of computerized physiologic measurements are available commercially for monitoring patients at risk of sudden death, including during surgery, in the post-surgical ICU, in the cardiac ICU, etc.

It is also well recognized that cardiopulmonary exercise testing (CPX) yields valuable information to quantify a patient's immediate physiologic condition in terms of aerobic capacity. A CPX system, of course, simply measures oxygen consumption (VO₂), carbon dioxide production (VCO₂), ventilation (VE), and heart rate (HR). Typically, from these measurements one can derive maximum aerobic capacity (peak attained VO₂) and an exhaustion index (anaerobic threshold, onset of respiratory compensation) of a patient.

A further multi-function CPX system is shown in Anderson et al (U.S. Pat. No. 4,463,754). That system includes a microprocessor-based waveform analyzer for performing real time breath-by-breath analysis of cardiopulmonary activity to measure a plurality of parameters including stress testing to for diagnosing and to ascertain physical fitness. While this device is an excellent source of evaluation data, it clearly does not function as a patient data management system for defining risk factors for specific patient populations. The use of such data in its raw form, consisting of tables and graphs of the measured data, is usually avoided by clinicians because the presentation of the data is incomplete and viewed as irrelevant to all but the most specialized clinician. There is simply too many data points and not enough translation of the data to tell the clinician what is needed: 1) what are the patient's risk factors, and 2) how is the patient responding to therapy over time? An additional limitation to CPX testing is the perceived need to exercise a patient to a valid peak VO₂. The method described herein utilizes cardiopulmonary slope variables (CSV) that are valid even when the patient fails to reach a peak VO₂, thereby shortening the total test time and thus, patient tolerance.

Further, there exists no similar computerized system for the long term monitoring of classes of data associated with patients with chronic diseases. Such diseases include chronic obstructive pulmonary disease COPD, congestive heart failure (CHF) due to hypertension or ischemic, coronary heart disease (CHD) or patients who may have cardiac pacemakers and/or implanted cardiac defibrillators for the treatment of brady and/or tachyarrhythmias or patients who may have peripheral vascular disease (PVD) resulting from atherosclerosis or deep vein thrombosis. A lengthy process of degeneration, as opposed to sudden death, characterizes these forms of chronic disease. Consequently, CHF is the most expensive of the diagnostic related groupings (DRG's) for medical reimbursement. Today, several therapies are available for treatment of patients with long term, chronic diseases, but the efficacy of such therapies is poorly understood due to the lengthy time required for these therapies to reverse the disease process and due to the lack of a fully integrated information feedback system to be used by the prescribing physician.

While the methods of the present invention, as described herein, provide a function similar to commercially available patient monitoring systems, several new classes of data are introduced, and these data classes are measured, translated, and presented for monitoring over a much longer time frame.

Another present drawback that further complicates the role of the physician is the lack of centralization of all relevant information available during treatment. Several classes of information that could be used to evaluate treatment exist, but these are currently provided as separate information sources. Blood samples are frequently collected to evaluate biochemical/neurohumoral data, such as brain natriuretic peptide (BNP) or C-reactive protein. The present invention reduces this complication by centralizing the data management function for multiple classes of relevant data.

### SUMMARY OF THE INVENTION

The present invention, to a large extent, obviates all of the problems discussed in the foregoing. The present invention presents a different philosophical approach to managing and processing data collected from a plurality of classes of related variables for which there exists a mean value and accepted or presumed standard deviation and cutoff point (the variable value which indicates the onset of increased risk of death). The method involves translating the data into statistically usable form and thereafter assigning magnitude values selected from positive and negative magnitude values and presenting the data as objects having a relative visualized value. Positive and negative values may be accumulated in a balance-type presentation, for example, to portray data weight.

In the detailed embodiment, patient data of dynamic and static varieties are used to illustrate the concept. The data is collected over an extended period of time to evaluate a patient's response to therapy. The invention includes several new evaluation concepts, including the integration of two classes of data variables: 1) dynamic-cardiopulmonary (DCP), and 2) static-biochemical/neurohumoral (SBN). The invention further describes the translation of raw DCP variables into breakpoints that define exhaustion thresholds and aerobic capacity and which are then displayed using a “virtual barometer” along with the normal values for the measured breakpoints. The raw DCP variable pairs are further translated into a cardiopulmonary slope variable (CSV)—a non-invasively measured variable that represents a surrogate measurement of one particular aspect of the performance of a patient's cardiovascular reflex control.

As will be described, the difference between a measured breakpoint, CSV and/or a SBNV and its statistically derived mean value is divided by the statistically derived Standard Deviation to define a new variable called an Autonomic Balance Index, (ABI). A “normalizing value” (NV) is also defined as the fractional number of standard deviations that the cutoff point differs from the mean value. An intermediate Mortality Prediction Index (MPI1) is then calculated by subtracting the ABI from the NV and further dividing this value by the NV. A final step in calculating the MPI is dividing the MPI1 by the NV.

Each MPI is “loaded” onto a “virtual balance beam scale”, whose “indicator” is designed to define whether the patient has an elevated risk of death and the relative magnitude of the risk. Negative values of MPI are loaded onto the left side of the scale and represent sympathetic overdrive and quantify patient risk of death. Positive values of MPI are loaded onto the right side of the scale and represent autonomic balance with no statistically imputed risk of death. The MPI values on each side of the scale are “weighed” and added to produce a sum, and the sign and magnitude are used to define a cumulative MPI for the patient for a particular date and time.

Additionally, trend graphs of each breakpoint, CSV, SBNV, individual MPI, and the cumulative ABI can be plotted over time to reflect therapy-induced changes. In this manner, patient risk of death may also be displayed using a Kaplan-Meier Plot derived from the source publication for the variable statistics.

Two classes of ABI are described: 1) dynamic-cardiopulmonary (DCP) and 2) static-biochemical/neurohumoral (SBN). The RV of the DCP class are VO₂, VCO₂, VE, and HR are measured using a cardiopulmonary exercise (CPX) testing system while the patient exercises on an ergometer that has been programmed to increase the work rate linearly over a short period of time (forcing function). These RV's are further analyzed to determine kinetics and breakpoints that reflect upon the forcing workload function and the physiologic changes experienced by the patient.

RV's of the SBN class (SBNV) are obtained from available laboratory blood chemistry instrumentation and include brain natriuretic peptide (BNP) and C-reactive protein. The results of this analysis are compared to statistical normal values for individuals of similar anthropometric data using a display of a “virtual barometer”.

The RV's of the DCP class are further analyzed to determine a new class of variable defined as a “cardiopulmonary slope variable” (CSV). Such analysis includes a linear regression analysis of two RV's plotted against one another to derive the slope of the response. The value thus derived is then compared to the mean value (MV) of the slope for that set of RV's obtained from the scientific literature and stored in a look-up table for all breakpoints, CSV's, and SBNV's. The MPI for the CSV is calculated as described above.

Similarly, RV's from the DCP class are also successively analyzed to yield the breakpoints. The analysis continues to derive the MPI for the DCP. Additionally, trend graphs of each cardiopulmonary breakpoint, CSV, SBNV and the cumulative MPI can be plotted over time to reflect therapy-induced changes. Additionally, any individual MPI is derived from the scientific literature, and the means to access the source publication is provided for physician reference.

Advantages

Accordingly, a principal advantage of the present invention to provide an improved method of collection, translation, integration, presentation, and management of multiple data sets. The data may be medically related data used to identify patient risk and to monitor therapy induced responses over time. Initially, this includes a method that integrates the data acquisition and translation of two classes of data: 1) dynamic-cardiopulmonary (DCP), and 2) static-biochemical/neurohumoral (SBN).

The invention provides a new way to visually display the measured and normal values of breakpoints observed from the “raw variables” measured by CPX testing using a “virtual barometer”.

As a further advantage, the present invention provides a means for measuring a plurality of breakpoints, including (1) peak attained VO₂, (2) anaerobic threshold, (3) onset of respiratory compensation, and (4) maximum attained oxygen pulse (VO₂/HR). The aforementioned list of breakpoints can be expanded with new such breakpoints as they become available in the scientific literature.

The invention provides a new class of variable—a CSV—which is derived from a plurality of “raw variables” measured by CPX testing and that represent a measure of cardiovascular reflex control and a system for measuring a plurality of CSV's, including (1) the Ventilatory Efficiency (slope of VE/VCO₂ ), (2) Chronotropic Response Index (ratio of heart rate reserve used to metabolic reserve used), (3) Aerobic Power (slope of VO2/Work Rate), (4) Oxygen Uptake Efficiency (slope of VO2/log VE), and (5) Heart Rate Recovery (slope of heart rate/time after 1 minute of recovery from exercise). The system further accommodates expansion of the aforementioned list of CSV's with new such CSV's as they become available in the scientific literature.

The method of the invention has the ability to obtain a plurality of SBNV's, including (1) BNP, and (2) C-reactive protein and integrates SBNV's acquired from laboratory blood chemistry instrumentation. The system advantageously can accommodate new such SBNV's as they become available in the scientific literature.

The new method of the invention further enables integration of data disclosed in scientific publications regarding statistically derived normal values for a plurality of breakpoints, CSV's and SBN's and can provide access to the source publications for normal values for breakpoints, CSV's, and SBN's for physician reference.

Another characteristic of the present invention is the ability to compare each measured breakpoint, CSV and SBNV with the statistically derived mean value, standard deviation, and cutoff point for each to compute the Mortality Prediction Index.

The system is further characterized by new visual display techniques including a “virtual balance beam scale” which can be used to depict autonomic balance and patient risk of death.

The present invention may also present trend plots of the breakpoints, CSV's, SBNV's, and the individual and cumulative MPI.

Finally, the present invention uses the data to define patient risk of death expressed as a Kaplan-Meier plot with the measured variable(s).

### BRIEF DESCRIPTION OF THE DRAWINGS

In the drawings:

FIG. 1 is a schematic drawing that illustrates the functional components of a CPX testing system usable with the present invention;

FIG. 2 illustrates three phases of dynamic-cardiopulmonary data collection, namely rest, isotonic exercise and recovery along a time line;

FIG. 3 illustrates the Autonomic Balance Index (ABI) Translation process of the invention;

FIG. 4 is a plot of VE/VCO₂ showing the line of regression and its slope;

FIG. 5 illustrates the format of the Object Definition Table with entries for each of the variable classes used in the examples provided in the Detailed Description;

FIG. 6 is a plot showing 02 pulse (VO₂/HR) against time;

FIG. 7 illustrates the Mortality Prediction Index (MPI) calculation steps;

FIG. 8 illustrates the properties of the MPI;

FIG. 9 illustrates a virtual balance beam scale loading protocol;

FIG. 10 illustrates a virtual balance beam scale loaded pursuant to the protocol of FIG. 9;

FIG. 11 further illustrates a virtual balance beam scale with accumulative MPI—with the pointer indicating a value on the scale as to whether the patient exhibits balance or is unbalanced toward sympathetic overdrive;

FIG. 12 illustrates a measured versus normal barometer comparing the translated variables with statistically normal values for each further noting the change in the translated measurements between sets of measurements;

FIG. 13 illustrates a Kaplan-Meier plot as a predictor of heart failure mortality; and

FIG. 14 illustrates a trend graph showing changes in the slope of VE/VCO₂ over time and the mean value for the slope of VE/VCO₂.

### DETAILED DESCRIPTION

The following detailed description with respect to patient data is intended to be exemplary of a preferred method of utilizing the concepts of the present invention and is not intended to be exhaustive or limiting in any manner with respect to similar methods and additional or other steps which might occur to those skilled in the art. The following description further utilizes illustrative examples which are believed sufficient to convey an adequate understanding of the broader concepts of processing data from a plurality of classes of related variables to those skilled in the art and exhaustive examples are believed unnecessary.

As indicated above, one class of data, dynamic-cardiopulmonary (DCP), is obtained using physical exercise testing performed in accordance with a standardized workload protocol as the forcing function to elicit physiologic changes resulting from increasing amounts of workload. Such data can be viewed as a description of the primary “endpoint” for a wide variety of medical therapies—data describing how an individual is able to function in the physical world in terms of the physiologic changes that the individual experiences when engaged in the performance of physical work.

The physiologic changes are measured using a cardiopulmonary exercise testing system (CPX), and these measurements, or “raw variables” (RV=VO₂, VCO₂, VE, HR), are then translated in successive stages to: (1) breakpoints, defined in terms of anaerobic threshold, onset of respiratory compensation, peak VO₂, and peak O₂ pulse; (2) “cardiopulmonary slope variable” (CSV) (3) visual display using a “virtual barometer” of the measured breakpoint and CSV in relation to the mean value and standard deviation for the breakpoint and CSV, (4) a computation of a Mortality Prediction Index for the individual breakpoint and CSV (5) a summation of all such CSV's and breakpoints into a cumulative MPI using a “virtual balance beam scale”, and (6) a quantified risk of death using a Kaplan-Meier plot.

In doing so, the “raw variables” are translated from a form from which nothing (other than a simple value with a unit of measurement) can be implied to a form from which meaningful information (diagnostic and prognostic) can be derived (this individual's capacity for physical work is less than it should be for a normal person) and expressed in statistical terms derived from scientific studies that define the meaning of the term “normal”. By analogy, traffic safety laws are based upon the measurement of the speed of an automobile, not it's position at any point in time. It then follows that the “safety” of an individual from death from chronic disease should not be judged by the heart rate at any point in time, but rather, for example, the rate of change of the heart rate (speed) when measured against the work performed over time.

As a convenience to the physician to improve and centralize pertinent data to more completely assess patient condition, additional classes of patient information are made available. As an example, static-biochemical/neurohumoral variables (SBNV), can be collected from available laboratory blood chemistry instrumentation. For each SBNV, steps similar to 4 and 5 are taken to derive an MPI for this class. When breakpoints, CSV's and SBNV's are accrued and analyzed together, their power of patient risk prediction becomes even more pronounced.

In doing so, a physician is relieved from performing the data translation and integration necessary to derive a true, physiologic assessment of the patient's condition at any point in time. By also providing trend plots of the translated data over time, the physician can better understand the consequence of any given therapeutic action. By providing a closed-loop system of action (therapy) and physiologic response (to therapy), the quality of treating patient's with cardiac and cardiovascular disease will be increased and the cost reduced.

In order to convey the required detail, it is not believed necessary to explain the translation process for each individual breakpoint, CSV, or SBNV or to explain how all are individually used to produce the desired outputs—a “virtual barometer”, the translated variable, a “virtual balance beam scale” using the cumulative MPI, trend graphs for each individual breakpoint, CSV, SBNV, MPI, and a Kaplan Meier plot. To avoid unnecessary repetition, the method by which a single breakpoint, CSV, and SBN is translated to an MPI will be described in detail. The additional methods used to produce the intended outputs from the generated MPI will also be described in detail.

The data gathering aspect of the invention involves known techniques and analyses and it is the aspects of processing and combining the data in which the invention enables an observer to gain new and valuable insight into the present condition and condition trends in patents. Thus, in accordance with the preferred method, a cardiopulmonary exercise test (CPX) is performed for each data set. The performance of such a test is well understood by individuals skilled in the art, and no further explanation of this is believed necessary. In addition, the measurement of the SBNV class of data is obtained by blood analysis using commonly available laboratory blood chemistry instrumentation in a well-known manner, and no further explanation of this procedure is believed required.

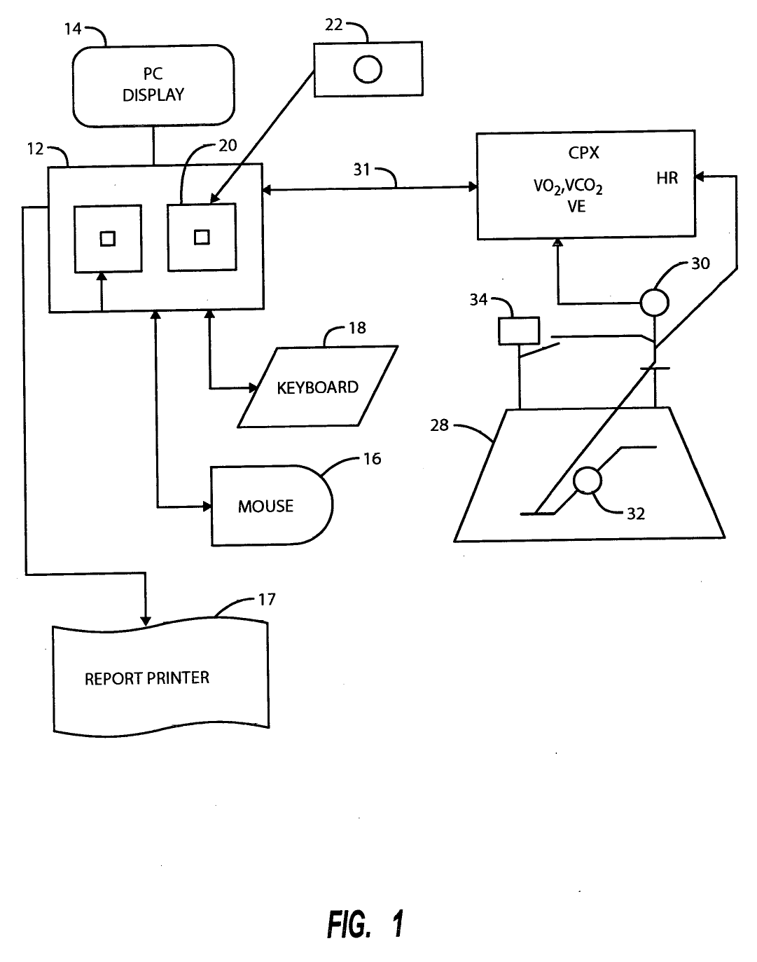

With this in mind typical hardware is shown in FIG. 1, which illustrates typical equipment whereby a cardiopulmonary exercise test (CPX) may be conducted and the results displayed in accordance with the method of the present invention. The system is seen to include a data processing device, here shown as a personal computer of PC 12 which comprises a video display terminal 14 with associated mouse 16, report printer 17 and a keyboard 18. The system further has a floppy disc handler 20 with associated floppy disc 22. As is well known in the art, the floppy-disc handler 20 input/output interfaces comprise read/write devices for reading prerecorded information stored, deleting, adding or changing recorded information, on a machine-readable medium, i.e., a floppy disc, and for providing signals which can be considered as data or operands to be manipulated in accordance with a software program loaded into the RAM or ROM memory (not shown) included in the computing module 12.

The equipment used in the protocol includes a bicycle ergometer designed for use in a cardiopulmonary stress testing system (CPX) as is represented at 28 together with a subject 30 operating a pedal crank input device 32. A graphic display device 34 interfaces with the subject during operation of the CPX device. Data in the form of stress dependent physiological and psychological variables are measured. The physiological variables may be selected from heart rate (HR), ventilation (VE), rate of oxygen uptake or consumption (VO₂) and carbon dioxide production (VCO₂) or other recognized variables. Physiological data collected is fed into the computing module 12 via a conductor 31, or other communication device.

### Calculation of an Individual Mortality Prediction Index (MPI)

Dynamic—Cardiopulmonary Class (DCP)

Cardiopulmonary Slope Variables

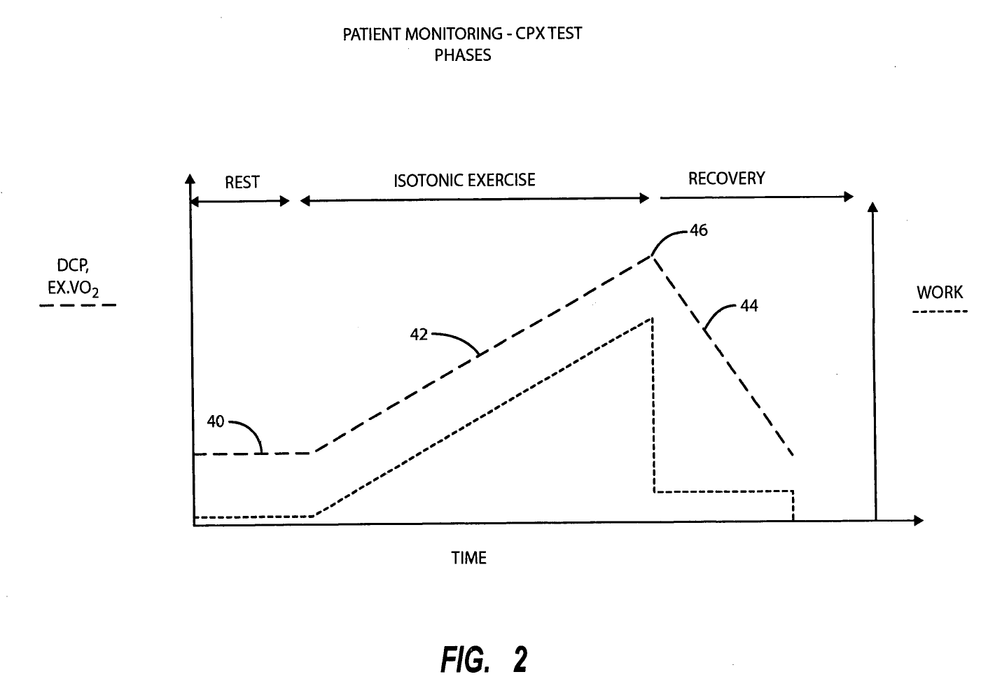

The raw DCP variables of VO₂, VCO₂, VE, HR, and are first measured using CPX testing while the patient exercises on an ergometer as shown in FIG. 1. This list is not intended to be all-inclusive or limiting, and, over time, additional such variables, such as blood pressure, will be included. As illustrated in FIG. 2, three phases of data collection are used, namely, rest 40, isotonic exercise 42, and recovery 44. It will be recognized that, because the raw DCP variables are translated into cardiopulmonary slope variables (CSV's), the patient is not required to exercise to exhaustion during the isotonic exercise phase. Instead, the exercise workload is terminated at 46 due to 1) patient fatigue, or 2) sudden acceleration of VE relative to VO₂ and VCO₂. The raw DCP variables are measured and collected for a predetermined amount of time after the workload has been removed (recovery period).

The raw DCP variables are then translated into one or more class of CSV. Initially, CSV's include: (1) the Ventilatory Efficiency (slope of VE/VCO₂), (2) Chronotropic Response Index (ratio of heart rate reserve used to metabolic reserve used), (3) Aerobic Power (slope of VO2/Work Rate), (4) Oxygen Uptake Efficiency (slope of VO2/log VE), and (5) Heart Rate Recovery (slope of heart rate/time after 1 minute of recovery from exercise). As previously stated, this list is not intended to be all-inclusive, and it is expected that additional such CSV's will become available from the scientific literature over time.

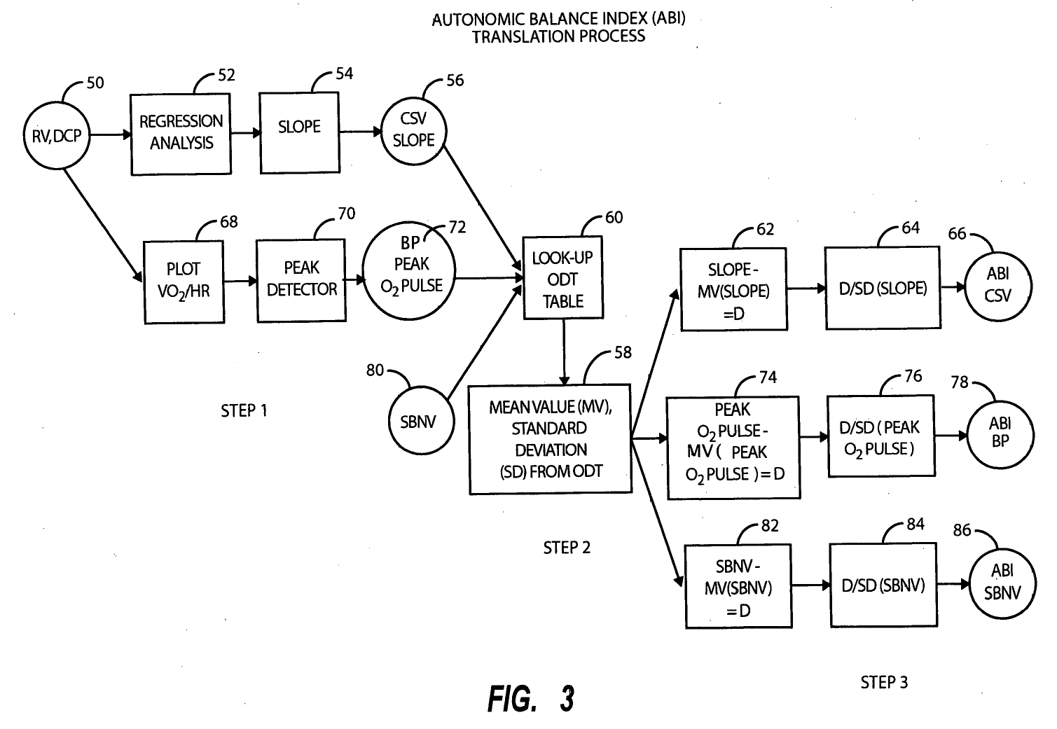

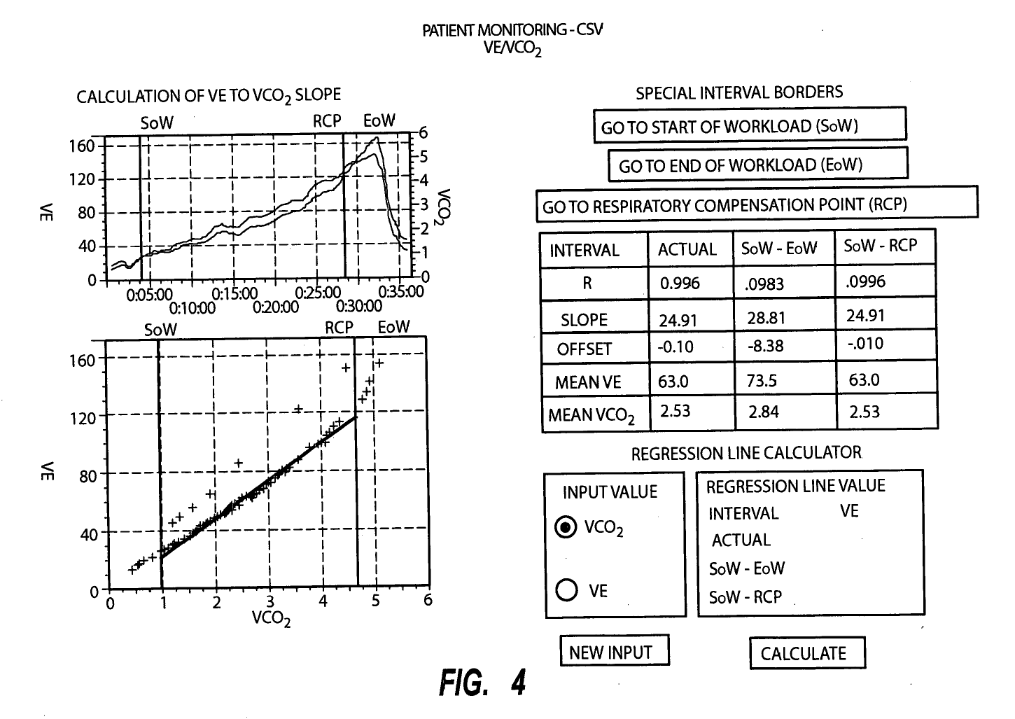

The first step in the preferred translation method is the execution of a computer program (FIG. 3). In Step 1, a linear regression analysis of two raw variables or RV's from 50 plotted against one another is performed at 52 to derive the slope 54 of the response illustrated in FIG. 4, using as an example, VE/VCO₂. The Cardiopulmonary Slope Variables (CSV) slope is also determined at 56 using regression analysis. With respect to the regression analysis, it will be noted that the recorded test data contain the channels minute ventilation VE and carbon dioxide output VCO₂ as time series with sample points (moments of time) tᵢ, so there are two sets of data points VEᵢ and VCO₂ᵢ with i−l, . . . , N. To find the best straight line fit VE=a VCO2+b to the ensemble of point pairs (VEᵢ, VC02ᵢ) one can use the linear regression analysis minimizing the sum of squares of distances of these points to a straight line, see for instance PRESS, W. H., B. P. FLANNERY, S. A. TEUKOLSKY, W. T. VETTERLING:; Numerical Recipes, The Art of Scientific Computing. Cambridge University Press, Cambridge etc., 1986, Chapter 14.2. The main results of such an analysis are the constants a and b describing the regression line and the regression coefficient r as a measure for the regularity of data lying along and around this line. The constant a is the VE to VCO₂ slope of the above mentioned data ensemble.

Not all recorded data are significant for the determination of the VE to VCO₂ slope parameter, but only that part of them belonging to the isotonic exercise phases (FIG. 2, at 42) of a CPX test.

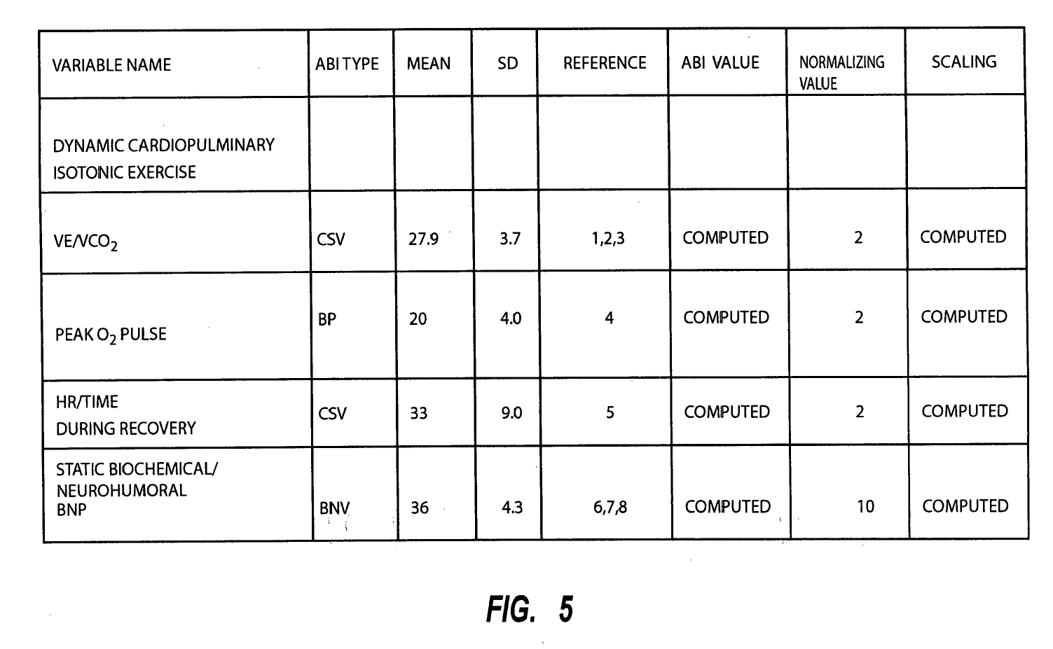

In Step 2 (FIG. 3), the mean value (MV) and standard deviation (SD) for the test subject is obtained at 58 from a look-up Object Definition Table 60 (see also FIG. 5). All translated variable types have an entry in the Object Definition Table. In FIG. 3, Step 3, the difference between the measured CSV and the MV is computed at 62, and the value thus derived is divided by the standard deviation of the CSV at 64(obtained from the aforementioned look-up table at 60) to yield a new variable defined as the Autonomic Balance Index for the CSV VE/VCO₂ slope at 66.

Breakpoints

After the CPX testing is finished, a computer program is executed to further analyze the raw DCP variables to determine the breakpoints (BP) that reflect upon the forcing workload function and the physiologic changes experienced by the patient during the isotonic exercise period. Certain BP's derived from the DCP class can be further translated into ABI values similarly to CSV's as described above.

Similar statistical information exists in the scientific literature, and such BP's include (1) peak attained VO₂, (2) maximum attained oxygen pulse (VO₂/HR), (3) anaerobic threshold, (4) onset of respiratory compensation (RC). This list is not intended to be all-inclusive, and it is expected that additional such BP's will become accepted standards in the scientific literature.

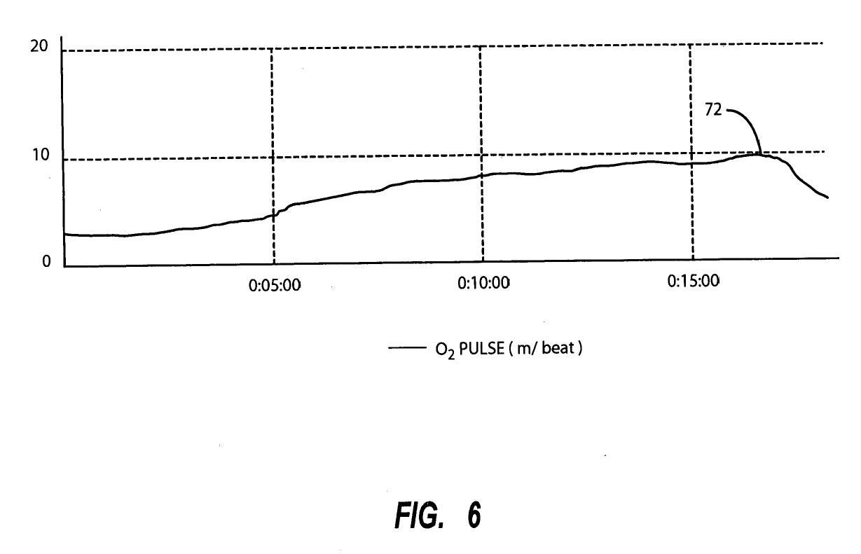

In a process similar to that described above for CSV's, a computer program (FIG. 3 at 50, 52 and 54) is executed at 68, 70 and 72. In Step 1, an analysis of 0₂ Pulse (VO₂/HR) is made to derive the BP. It uses FIG. 6 as an example, the plot of 0₂ Pulse against time is shown at 68 for detecting the peak value at 70. The peak 0₂ Pulse is shown at 72. In Step 2, the mean value (MV) and standard deviation (SD) for peak 0₂ Pulse is derived at 58 for the test subject 60 as was the case with the CSV variables and is obtained from the Object Definition Look-Up Table (FIG. 5). In FIG. 3, Step 3, the difference between the measured peak 0₂ Pulse and the MV is computed at 74. The value thus derived is divided by the standard deviation of the peak 0₂ Pulse at 76 to yield a new variable defined as the Autonomic Balance Index (ABI) for the BP variable peak 0₂ Pulse at 78.

Static-Biochemical/Neurohumoral Class (SBN)

The raw SBNV, shown at 80 in FIG. 3, is measured as indicated previously. Initially, SBNV's include: (1) BNP, and (2) C-reactive protein. This list is not intended to be all-inclusive or limiting, and it is expected that additional such SBNV's will become available from the scientific literature over time.

In a process similar to that described above for CSV and BP, a computer program (FIG. 3, Steps 1-3) is executed. In Step 2, the mean value (MV) and standard deviation (SD) for the SBNV 80 for the test subject is also obtained at 58 from the Object Definition Table at 60. In Step 3, the difference between the measured SBNV and the MV is computed at 82, and the value thus derived is divided by the standard deviation of the SBNV at 84 (obtained from the aforementioned look-up table 60) to yield a new variable defined as the Autonomic Balance Index (ABI) for the SBNV at 86.

Calculating the MPI

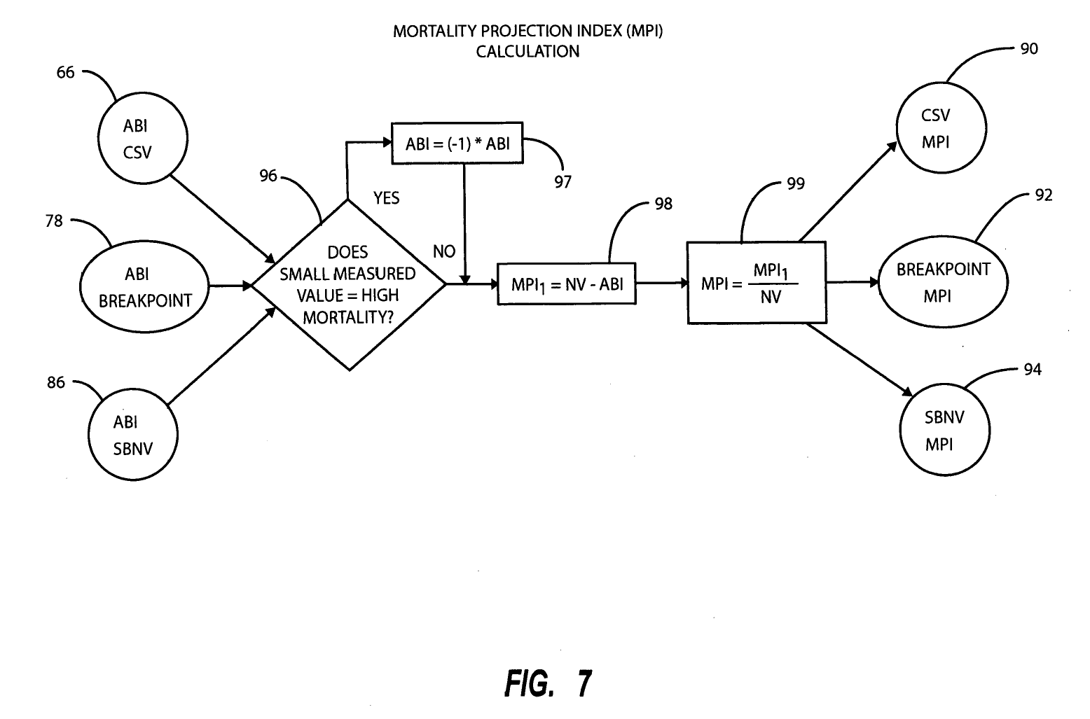

The next step in the preferred translation method, a computer program (FIG. 7) is executed to define an MPI whose properties are defined in the Object Definition Table (FIG. 5). The concept of the Normalizing Value (NV) allows us to further translate the ABI. The NV links the measured value for the CSV, BP, or SBN to the research data defining patient risk of death. The NV is a number that, when the ABI is subtracted from it, yields a value this indicative of elevated risk. The value of MPI=(NV−ABI)/NV at 98, and, by definition, a negative value indicates elevated risk. A mitigating factor is that some variables (ventilatory efficiency slope) have high values indicating high risk. Some (chronotropic response index) have low values indicating high risk. For this reason, the sign of the ABI must be adjusted accordingly, at 96. The more negative the MPI value is, the greater the risk of death. A positive MPI simply indicates that the translated value of the measured variable is outside the range of elevated risk as defined by the cutoff point.

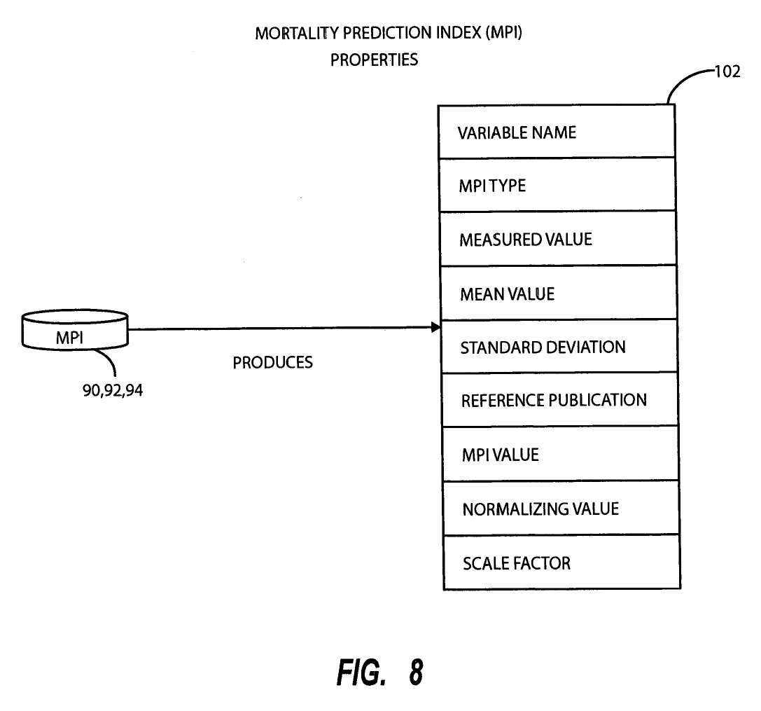

The calculated MPI values for CSV, Breakpoint, and SBNV are then computed at 90, 92, 94 for a particular corresponding ABI 66, 78, at 86. As depicted in FIG. 8, when a user “right-clicks” the system mouse at 100, the MPI properties are displayed in a drop-down list 102.

Loading and Displaying the Virtual Balance Beam Scale with MPI

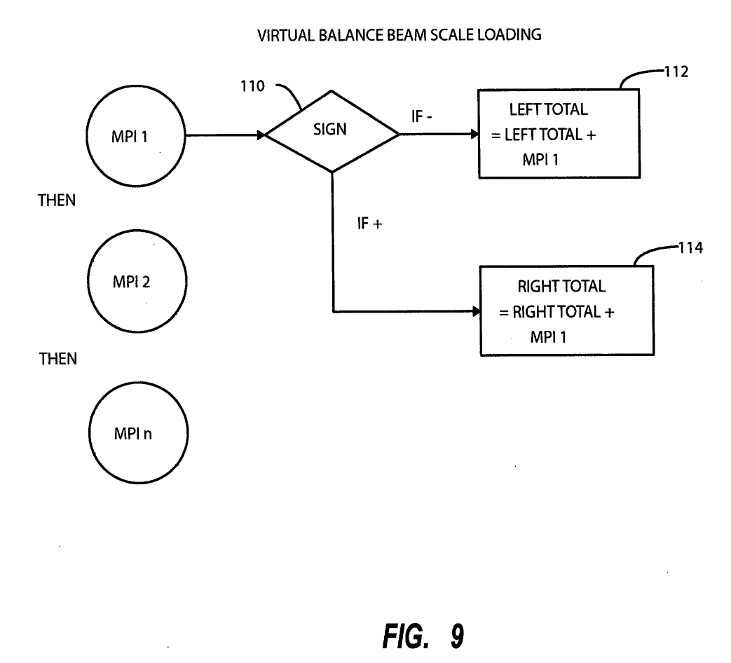

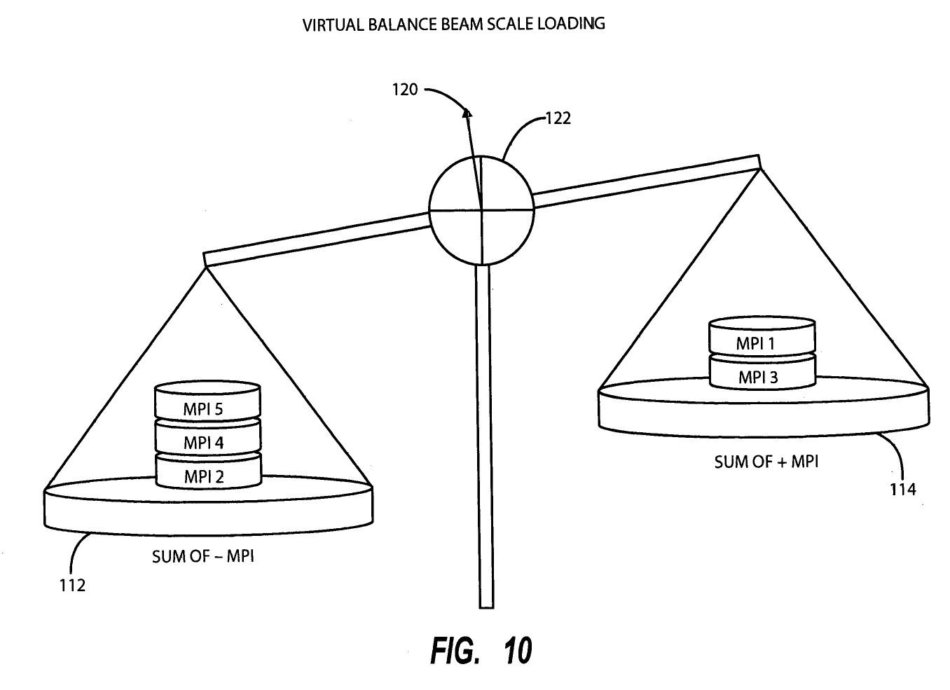

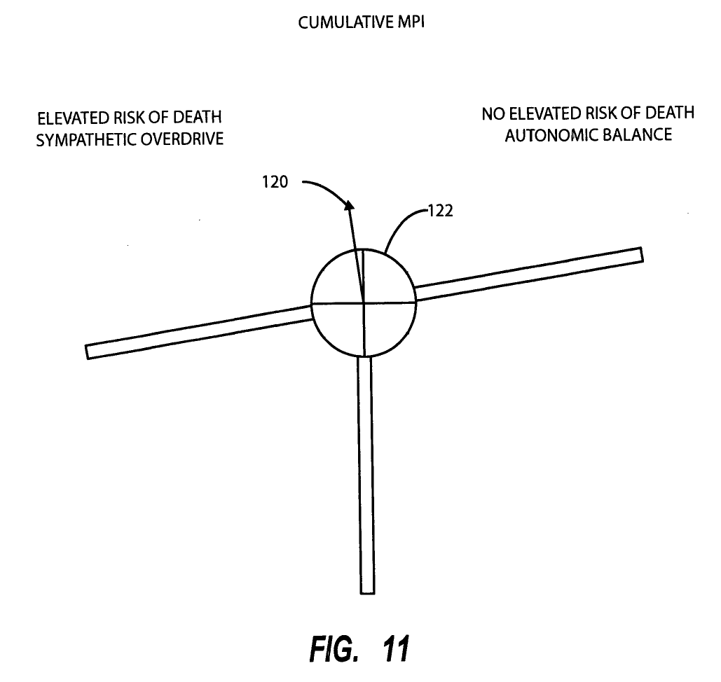

The next step in the illustrative translation method is the execution of a computer program to display a “virtual balance beam scale” loaded with the MPI whose values have been computed as above. Each previously defined MPI is processed in FIG. 9. If the sign of the MPI at 110 is negative (indicating sympathetic overdrive), the MPI is “loaded” onto the left side of the scale at 112. If the sign at 110 of the MPI is positive (indicating autonomic balance), the MPI is “loaded” onto the right side of the scale and becomes part of a cumulative total at 114. Upon completion of this process, all of the MPI that are “left loaded” will appear on the left scale pan, and all of the MPI that are “right loaded” will appear on the right scale pan. An example of a loaded balance beam scale will appear as in FIG. 10. The “virtual pointer” 120 will then indicate a value on the scale 122 and whether the patient exhibits autonomic balance or is unbalanced toward sympathetic overdrive and elevated risk of death and is shown relatively at FIG. 11.

Preferred Method for Displaying the Virtual Barometer

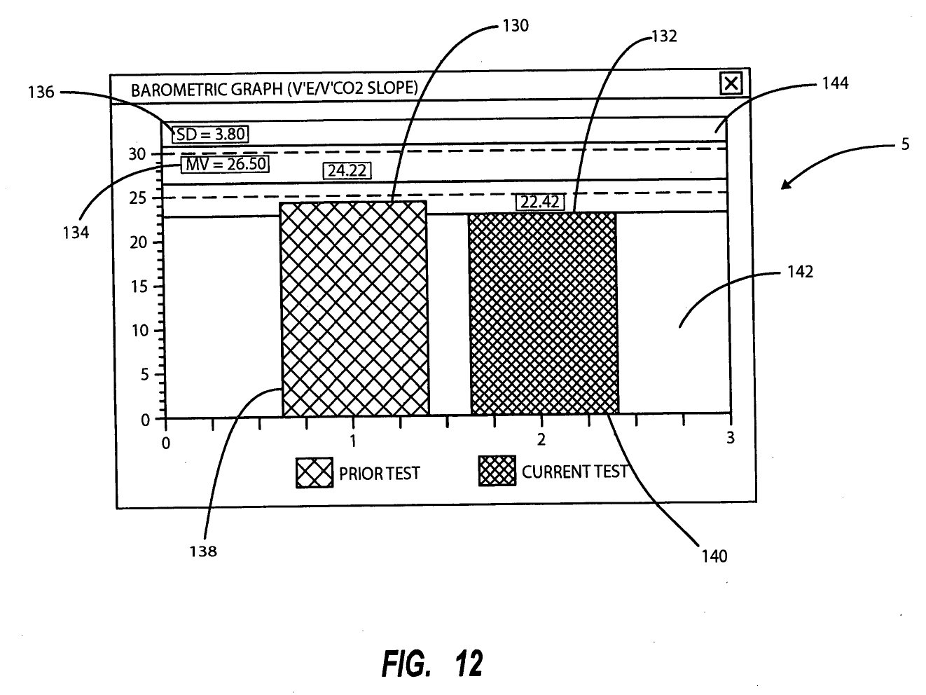

In FIG. 12, the translated measurements as shown at 130, 132 and the statistical mean value and standard deviation are then displayed at 134, 136 on a “virtual barometer”, thereby providing a graphical depiction of the patient's status in relationship to a “normal” individual. The barometer is represented as a bar 138 whose height equals the measured variable. Subsequent test values can be displayed at 140 for comparison purposes. In addition, the areas below and above one standard deviation can be color coded to indicate whether the measured variable represents an improvement in the patient's status (green shading at 142) or a deterioration in the patient's status (red shading at 144). In this manner trend information can be derived as well

Displaying the Risk of Death

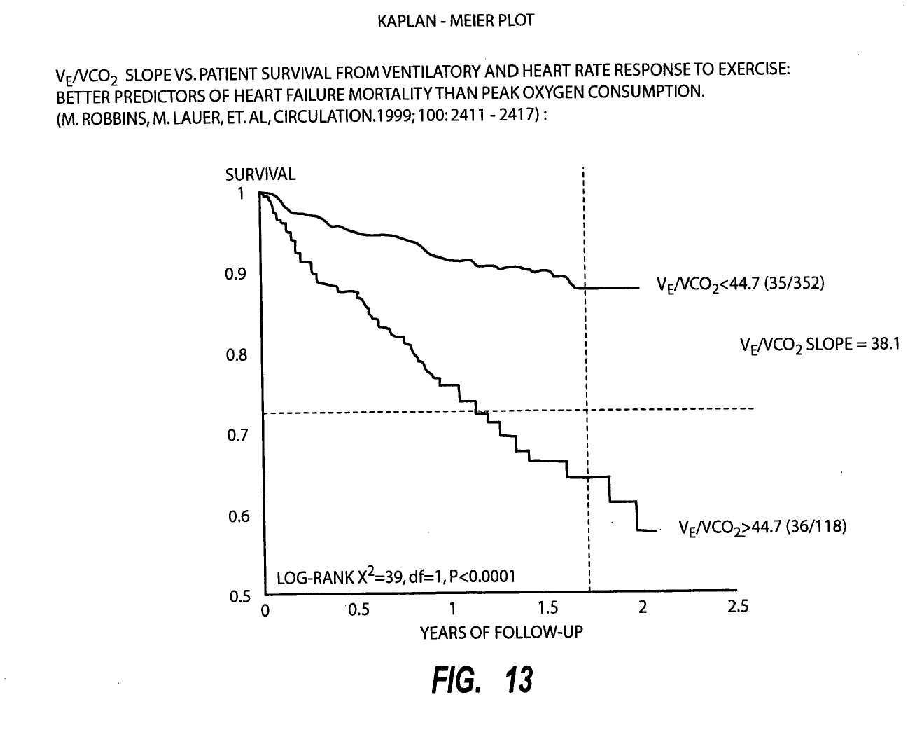

The patient risk of death is displayed using a Kaplan-Meier plot as illustrated in FIG. 13. The value of the translated variable and the source publication are printed on a reproduced plot, as depicted in FIG. 13.

Preferred Method for Displaying Trend Graphs.

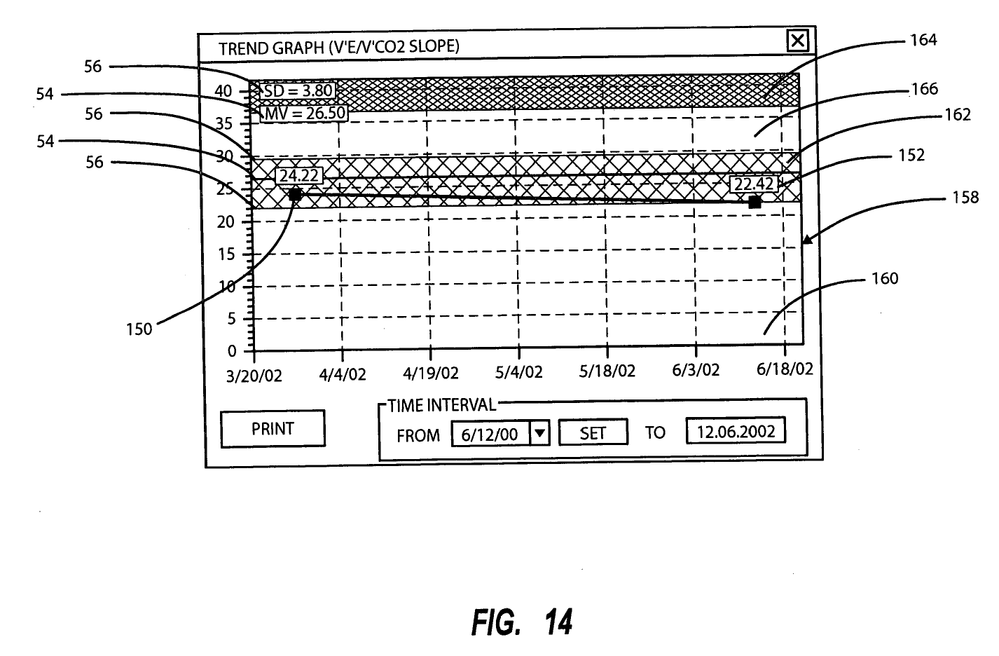

The next step in the preferred translation method is to provide trend graphs of the measured variables, individual MPI, and cumulative MPI for successive testing dates (FIG. 14). In FIG. 14, the measurements as shown at 150, 152 and the statistical mean value and standard deviation for each are then displayed at 154, 156, thereby providing a graphical depiction of the patient's status in relationship to a “normal” individual. The cutoff point is displayed at 158. Thus, separate zones are defined: below the mean less one standard deviation 160, the mean value plus and minus one standard deviation 162, and the area beyond the cutoff point 164. In FIG. 14, another zone can be shown at 166 which is the area above one standard deviation and the cutoff point (this also illustrates the difference between the terms “cutoff point” and “standard deviation”). In addition, the areas below one standard deviation 160 above the cutoff point 164 can be color coded to indicate whether the measured variable represents an improvement in the patient's status (green shading at 160) or a deterioration in the patient's status (red shading at 164).

The invention has been described in considerable detail in order to comply with the Patent Statutes and to provide those skilled in the art with the information needed to apply the novel principles and to construct and use such specialized components as are required. However, it is to be understood that the invention can be carried out by specifically different equipment and devices, and that various modifications, both as the equipment details and operating procedures can be accomplished without departing from the scope of the invention itself.

### REFERENCES

1. J. Laukkanen, J. T. T. Salonen, et al., Association of Maximum Oxygen Pulse during Exercise Stress Test with the Risk of Cardiovascular and Overall Mortality. J. Amer. College Cardiol. 2002;39(5): Abst #3832)

2. M. Robbins, M. Lauer et al., Ventilatory and Heart Rate Response to Exercise: Better Predictors of Heart Failure Mortality Than Peak Oxygen Consumption, Circulation, 1999; 100:2411-2417

3. F X Kleber, S. Glaeser et al., Impairment of Ventilation Efficiency in Heart Failure: Prognostic Impact, Circulation, 2000; 101; 2803-2809

4. P. Ponikowski, Anker et al., Enhanced Ventilatory Response to Exercise in Patients with Chronic Heart Failure and Preserved Exercise Tolerance: marker of Abnormal Cardiorespiratory Reflex Control and Predictor of Poor Prognosis, Circulation, 2001; 103;067-972

5. C. Cole, M. Lauer, Heart Rate Recovery Immediately Following Exercise as a Predictor of Mortality, New England Journal of Medicine, Vol. 341, (18) 1351-1357, 1999

6. Q Dao, A. Maisel et al., Utility of B-Type Natriuretic Peptide in the Diagnosis of Congestive Heart Failure in an Urgent-Care Setting, J Am Coll Cardiol, 2001; 37:379-385

7. E. Lubien, A. Maisel et al., Utility of B-Natriuretic Peptide in Detecting Diastolic Dysfunction: Comparison with Doppler Velocity Recordings, Circulation, 2002; 105:595-601

8. A. Maisel, A. DeMarta et al., Utility of B-Natriuretic Peptide as a Rapid, Point-Of-Care Test for Screening Patients Undergoing Echo-Cardiography to Determine Left Ventricular Dysfunction, Am Heart J., 2001; 141:367-374

What is claimed is:

1. A method of assessing therapy provided to a patient with chronic cardiovascular or cardiopulmonary disease including the step of graphically displaying individual and cumulative risk of death analysis based on selected risk factors derived from physiological measurements translated and combined mathematically into visual, virtual objects.

2. A method as in claim 1 wherein the selected risk factors are derived from measurements selected from the group consisting of dynamic, cardiopulmonary exercise testing variables and from static, biochemical/neurohumoral variables and combinations thereof.

3. A method as in claim 2 wherein the risk factors are derived from measurements that include both dynamic cardiopulmonary exercise testing variables and static biochemical/neurohumoral variables.

4. A method as in claim 2 wherein said dynamic, cardiopulmonary exercise testing variables of said physiological measurements are translated into a class of variable known as an autonomic balance index using the following steps:
(a) creating a first translation of dynamic, cardiopulmonary variables by performing a linear regression analysis to yield a slope of the line of regression;
(b) creating a second translation of dynamic, cardiopulmonary variables by performing a breakpoint analysis to yield a numeric value;
(c) defining an object definition table containing the statistically derived values for mean, standard deviation, and normalizing value for the intermediate values obtained in steps (a) and (b) above;
(d) subtracting the mean values obtained from the object definition table from the measured values obtained in steps (a) and (b) above to obtain a difference;
(e) dividing the difference obtained in step (d) by the standard deviation obtained from the object definition table.

5. A method as in claim 2 wherein said static, biochemical/neurohumoral variables of said physiological measurements are translated into a class of variable known as an automatic balance index using the following steps:
(a) making static measurements of one or more biochemical/neurohumoral variables
(b) defining an object definition table containing the statistically derived values for mean, standard deviation, and normalizing value for the values obtained in making said static measurements;
(c) subtracting the mean values obtained from the object definition table from the values of said static measurements obtained above to obtain a difference;
(d) dividing the difference obtained in step (c) by the standard deviation obtained from the object definition table.

6. A method as in either of claims 4-5 wherein the autonomic balance index obtained is further translated into a mortality prediction index (MPI) that can also be represented as a visual object that can quantify and typify an individual risk factor according to additional steps of:
(f) inverting the sign of the autonomic balance index previously obtained in claims 4 and 5 if a small number is indicative of higher risk
(g) subtracting the value obtained in (f) from a normalizing value, representing fractional number of standard deviations that the cutoff value differs from the mean value
(h) further dividing the result obtained in (g) by the normalizing value to yield the final value for the MPI; and
(i) scaling the visual object to a size proportional to the value obtained in (h).

7. A method as in claim 6 wherein individual physiologic risk factors, are mathematically combined and displayed using a “virtual” weighing apparatus, comprising the further steps of:
(i) accumulating the individual values of those visual objects having a negative sign using a one or more mathematical operators into a new value;
(j) accumulating the individual values for those visual objects having a positive sign, accumulate the individual values using one or more mathematical operators into a new value;
(k) placing the visual objects with a negative sign on the left pan of a 2-pan balance beam scale;
(l) placing the visual objects with a positive sign on the right pan of a 2-pan balance beam scale;
(m) causing the indicator of the balance beam scale to point to a scale value equal to the difference between the new values determined in (i) and (j) and tip the balance beam at an angle from horizontal that is proportional to this difference, one direction if positive, another direction if negative;
(m) define a region in which the indicator is pointing to the left side of 0 as elevated risk of death; and
(n) define a region in which the indicator is pointing to the right side of 0 as no elevated risk of death.

8. A method as in either of claims 4 or 5 wherein the translated variables are displayed in relationship to the statistical mean values and standard deviations using a “virtual barometer”.

9. A method as in either of claims 4 or 5 wherein the translated variables are displayed along with a Kaplan-Meier plot with shading designed to show positive or negative results.

10. A method as in either of claims 4 or 5 wherein the translated variables are displayed as time-sequential graphs and related to their mean values, standard deviations, cutoff points, and shading designed to show positive or negative trends.

11. A method as in claim 6 wherein the translated variables and their mortality prediction indices are displayed as time-sequential graphs.

12. A method as in claim 7 wherein the translated variables and their mortality prediction indices are displayed as time-sequential graphs.

13. A method as in either of claims 4-5 wherein said dynamic cardiopulmonary exercise testing variables are obtained without maximum effort by the patient.

14. A method of processing data comprising steps of:
(a) gathering data from a plurality of classes of related variables; wherein there exists a mean value and a standard deviation;
(b) translating said data into statistically usable form;
(c) assigning magnitude values selected from positive and negative values to and presenting said data as objects having a relative visualized value.

15. A method as in claim 14 further comprising the step of accumulating said objects on a scale to produce a net indicated result.

16. A method as in claim 15 wherein said objects are accumulated as weights on a virtual balance beam scale.

17. A method of presenting data for assessing therapy provided to a patient with chronic cardiovascular or cardiopulmonary disease including the step of graphically displaying individual and cumulative risk of death analysis data based on selected risk factors derived from physiological measurements translated and combined mathematically into visual, virtual objects.

18. A method as in claim 17 wherein the selected risk factors are derived from measurements selected from the group consisting of dynamic, cardiopulmonary exercise testing variables and from static, biochemical/neurohumoral variables and combinations thereof.

19. A method as in claim 18 wherein the risk factors are derived from measurements that include both dynamic cardiopulmonary exercise testing variables and static biochemical/neurohumoral variables.

20. A method as in any of claims 17-19 wherein said physiological measurement data pertain to one or more of said risk factors presented as a class of variable known as an autonomic balance index.

21. A method as in claim 20 further comprising the step of translating and presenting said autonomic balance index as a mortality prediction index in the form of a visual object that can quantify and typify an individual risk factor.

22. A method as in claim 21 wherein the translated variables and their mortality prediction indices are displayed as time-sequential graphs.

23. A method as in claim 21 wherein the translated variables are displayed along with a Kaplan-Meier plot with shading designed to show positive or negative results.

24. A method as in claim 21 wherein the said translated variables are displayed in relationship to the statistical mean values and standard deviations using a “virtual barometer”.

25. A method as in claim 17 further comprising the step of accumulating said objects on a scale as a visual display to produce a net indicated result.

26. A method as in claim 25 wherein said objects are represented as weights on a virtual balance beam scale.
